(19) United States
(12) Patent Application Publication
(10) Pub. No.: US 2022/0265922 A1
(43) Pub. Date: Aug. 25, 2022

(54) INTRAVENOUS SET TIMER DEVICE

(71) Applicant: CareFusion 303, Inc., San Diego, CA (US)

(72) Inventor: Leyla YAMIN, San Diego, CA (US)

(21) Appl. No.: 17/590,132

(22) Filed: Feb. 1, 2022

Related U.S. Application Data
(60) Provisional application No. 63/153,283, filed on Feb. 24, 2021.

(51) Int. Cl. A61M 5/162; A61M 5/168

(52) U.S. Cl. CPC A61M 5/162; A61M 5/1689; A61M 2205/584; A61M 2207/00

(57) ABSTRACT

Intravenous set timer devices are provided that include an indicator portion configured to change color at one or more predetermined times upon activation, a legend portion, wherein the legend portion defines relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion. Intravenous sets and methods of making an intravenous set with an intravenous set timer device are also provided.

### CROSS-REFERENCE TO RELATED APPLICATION

This application claims the benefit of priority under 35 U.S.C. § 119 to U.S. Provisional Patent Application Ser. No. 63/153,283, entitled “INTRAVENOUS SET TIMER DEVICE,” filed on Feb. 24, 2021, the entirety of which is incorporated herein by reference.

### TECHNICAL FIELD

The present disclosure generally relates to intravenous (IV) set devices, in particular IV set timer devices.

### BACKGROUND

Typical infusion or intravenous (IV) sets are constructed by joining multiple translucent polymeric tubing segments to multiple polymeric components, many of which are also translucent. These IV sets are then used with infusion pumps or gravity systems to provide fluids to a user, such as a patient. However, an IV set typically has both a predetermined shelf life (e.g., 3-5 years storage time) and a predetermined use life (e.g., 24 hours, 72 hours, 96 hours, 7 days). In particular, once an IV set is removed from its packaging and put into use, the IV set should only be used for a length of time up to the use life, after which time the IV set should be changed out. The time of use may be determined by healthcare regulations, the type(s) of drugs, medications or substances that are being infused through the IV set, and the types of materials used to make the IV set.

Currently, there is no good way to alert the clinician when an IV line should or needs to be changed. Typically, when the IV set is put into use, it is up to the clinician to chart or note the start time of the IV set use. However, in the hectic environment of some medical settings, the clinician may not have time to chart or note the start time. In addition, it may not be clear which use life time period a particular IV set in use requires. Further, when a clinician does determine that an IV set change is required, the action of changing the IV set is time and labor intensive, requiring locating the set, determining the medication being provided by the set and manually labeling IV lines. If the patient has several IV lines, such as in the intensive care unit (ICU), the task of finding and changing out the correct IV set(s) can be time consuming.

For these reasons, it is desirable to provide devices and methods for providing IV sets with built-in timers that visually show how much life is left of the associated IV set, thus giving the clinician the ability to determine the remaining use life of any IV set and to provide ample time for the clinician to make preparations for changing the IV set.

### SUMMARY

In one or more embodiments, an intravenous set timer device includes an indicator portion configured to change color at one or more predetermined times upon activation, a legend portion, wherein the legend portion defines relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion.

In one or more embodiments, an intravenous set includes a plurality of connected intravenous set components and an intravenous set timer device. The intravenous set timer device includes an indicator portion configured to change color at one or more predetermined times upon activation, a legend portion, wherein the legend portion defines relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion.

In one or more embodiments, a method of making an intravenous set with an intravenous set timer device includes attaching the intravenous set timer device to an intravenous set component, wherein the intravenous set timer device comprises an indicator portion configured to change color at one or more predetermined times upon activation, a legend portion defining relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion. The method also includes assembling a plurality of intravenous set components, including the intravenous set component with the attached intravenous set timer device, into an assembled intravenous set and packaging the assembled intravenous set.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIGS. 2A-2C are top views of example IV sets and extension sets.

FIG. 3 is a front view of an IV set timer device, according to aspects of the disclosure.

FIG. 4 depicts a perspective view of an IV set with timer device, according to aspects of the disclosure.

FIG. 5 depicts a perspective view of an IV set with timer device, according to aspects of the disclosure.

FIG. 6 depicts a perspective view of an IV set with timer device, according to aspects of the disclosure.

FIG. 7 depicts a perspective view of an IV set with timer device, according to aspects of the disclosure.

FIG. 8 illustrates a method of making an IV set with an IV set timer device, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

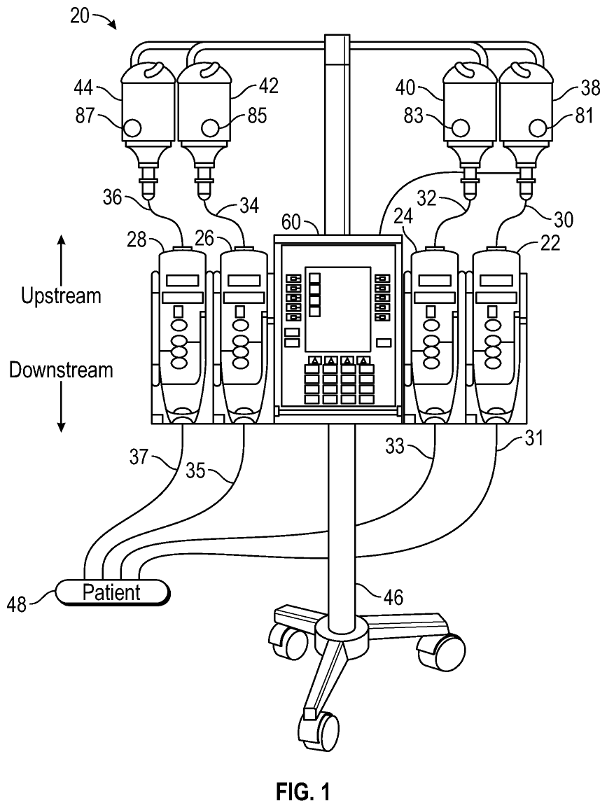

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

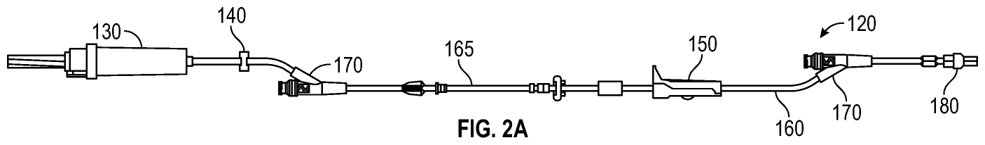

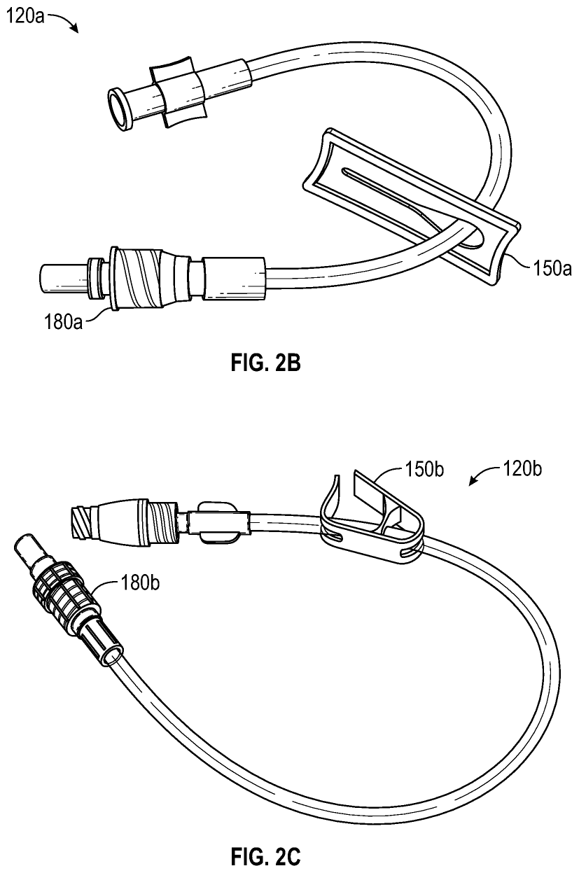

Typically, medical fluid administration sets have more parts than are shown in FIG. 1, such as those shown in FIGS. 2A-2C. Infusion sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2A, an infusion set 120 may include a drip chamber 130, a check valve 140, a flow controller 150 (e.g., roller clamp) and a pump segment 165 connected together by tubing 160. The infusion set 120 may also include a Y-site 170 having a Y-shaped junction with a needleless port 175, as well as a luer lock connector 180 at the end of the infusion set 120. The luer lock connector 180 may be used for connection to a catheter inserted into a patient, for example. The infusion set 120 may include additional infusion components and may be formed of any combination of components and the tubing 160. For example, substituting a length of tubing 160 for the pump segment 165 of infusion set 120 can change the infusion set 120 from a pump IV set to a gravity IV set.

As shown in FIG. 2B, an IV extension set 120a may be used to connect any two infusion components or devices, such as IV sets, infusion pumps, syringe pumps and the like. IV extension set 120a includes a luer lock connector 180a and a flow controller 150a. Similarly, as shown in FIG. 2C, another IV extension set 120b includes a luer lock connector 180b and a flow controller 150b.

In aspects of the disclosure, the subject technology provides a timer device as part of the IV set. For example, the IV set may have a built-in timer indicator that visually shows the remaining use life of the IV set. This provides for accurate use of IV sets, eliminating both too frequent changing of IV sets (e.g., clinician not sure when IV set use life is up and changes IV set too frequently, wasting the remaining use life hours of the replaced IV set) and overuse of an IV set past the use life of the IV set (e.g., clinician not sure when IV set use life is up and changes IV set too infrequently, possibly causing medical, administrative and legal issues).

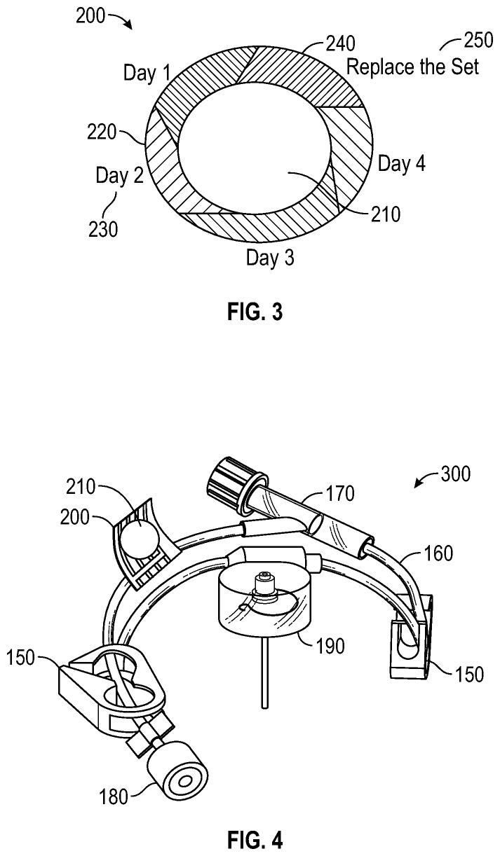

As shown in FIG. 3, an IV set timer device 200 is provided. IV set timer device 200 may be a label or a tag having a structure that changes color when exposed to carbon dioxide (CO₂), such as CO₂ in the ambient air where the IV set is being used. For example, the IV set timer device 200 may start timing when the IV set of which the IV set timer device 200 is associated is removed from its packaging (e.g., vacuum sealed), thus exposing the IV set timer device 200 to CO₂ in the air. As another example, the IV set timer device 200 may start timing when a cover is removed (e.g., peeled off), again exposing the IV set timer device 200 to CO₂ in the air.

As yet another example, the IV set timer device 200 may have a structure that changes color when exposed to a temperature change. For example, the IV set timer device 200 may be disposed on an infusion set component that changes temperature based on a temperature of an infusion fluid that enters the infusion set component when the IV set is put into use.

As shown in FIG. 3, the IV set timer device 200 includes an indicator portion 210, a legend portion 220, timing legends 230, an instruction portion 240 and an instruction legend 250. The indicator portion 210 as shown in FIG. 3 is the center circle portion, which changes color throughout the use life of the IV set. For example, in storage and prior to use, the indicator portion 210 may have a peel off label covering it and protecting the indicator portion 210 from exposure to CO₂ in the air. Accordingly, when the IV set is put into use, the peel off label may be removed and the subsequent exposure to CO₂ in the air starts the timer function of the IV set timer device 200.

The legend portion 220 as shown in FIG. 3 is an outer perimeter around a majority of the indicator portion 210. The legend portion 220 includes different colored sections that are each associated with a certain period of time (e.g., orange section, red section, light brown section, dark brown section). The associated time period is indicated by timing legends 230, such as word descriptions of the time period (e.g., day 1, day 2, day 3, day 4). For example, when the indicator portion 210 is activated by exposure to CO₂ in the air, the indicator portion 210 may already match or change to match the color of the legend portion 220 for the first time period (e.g., orange color associated with day 1). As each time period expires, the indicator portion 210 changes color to match that of the next time period (e.g., red color associated with day 2).

The instruction portion 240 as shown in FIG. 3 is the remaining portion of the outer perimeter around the indicator portion 210 and includes a different colored section than any of the legend portions 220 (e.g., yellow section). An associated instruction legend 250 may be a word and/or picture based instruction (e.g., replace the IV set) as to what is to be done when the use time of the IV set is expired. For example, when the final time period expires, the indicator portion 210 changes color to match that of the instruction portion 240.

Thus, with a quick glance at the indicator portion 210 of an IV set timer device 200 for an IV set that is in use, a clinician can easily and quickly determine how much use life remains for the IV set. For example, if the indicator portion 210 shown in FIG. 3 is dark brown, the clinician can quickly determine that the IV set is within its last day of use life and can make preparations for replacing the IV set within that 24 hour period. Here, the clinician can put in an order for a replacement IV set, check the room or local inventory to make sure a replacement IV set is on hand, enter instructions for the change of shift that an IV set will need to be replaced soon, and any other preparations that may be necessary. In aspects of the disclosure, the indicator portion 210 allows the clinician to know if the device has enough usage time left prior to starting an infusion therapy that would exceed the life of the IV set. In this case, the clinician can change the IV set prior to starting the IV therapy to avoid having to interrupt the therapy to replace the IV set.

In aspects of the disclosure, an IV set timer device may have any suitable form or format. For example, instead of the circular form shown in IV set timer device 200, the IV set timer device may be a linear strip where sequential sections of the strip change color over time. As another example, the IV set timer device may be a strip, a circular patch, a square patch or any other geometric shaped patch where the entire strip or patch evenly changes color over time. For example, a geometric patch that starts changing color from a point in the center and the color change radiates outward to the perimeter of the geometric shape over time, where the color change reaching the perimeter indicates the use life of the IV set or IV set component is expired. Another example is a linear strip where the color change begins at one end and moves towards the other end over time, again indicating expiration of the use life time period when the color change reaches an end line on the strip.

In aspects of the disclosure, legends and/or instructions may be disposed on any suitable portion of the IV set timer device. For example, legends and/or instructions may be printed on top of the indicator portion where the color change occurs underneath and/or around the legend/instruction, may be printed on outer portions other than the indicator portion (e.g., past the perimeter or end line), formed as part of the indicator portion so that the legend/instruction becomes visible when that portion of the indicator portion changes color, or any other suitable form.

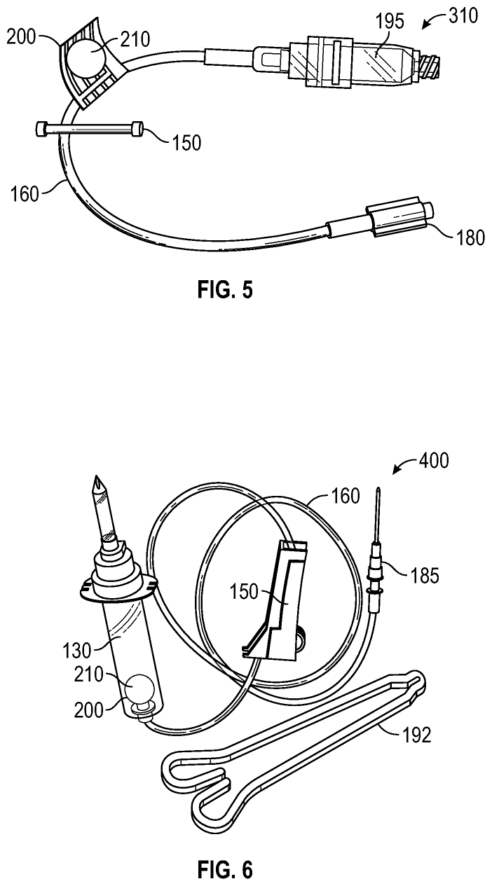

FIGS. 4 and 5 show example IV extension sets 300, 310 having different combinations of small infusion components and IV tubing 160. For example, extension set 300 includes a Y-site 170, a spike 190, a luer lock connector 180, IV tubing 160 and flow controllers 150, while extension set 310 includes a needless port 195, a flow controller 150, a luer lock connector 180 and IV tubing 160. Both IV extension sets 300, 310 include an IV set timer device 200 affixed to the IV tubing 160 as a label, each IV set timer device 200 having an indicator portion 210 that changes color over time after being activated. Here, the IV set timer device 200 may be affixed to the IV tubing 160 as a label if there are no infusion components within the IV extension set 300, 310 that are suitable (e.g., large enough, allowed to be partially obscured) to include the IV set timer device 200.

According to aspects of the disclosure, when the IV extension set 300, 310 is removed from vacuum sealed or airtight packaging, the indicator portion 210 may be automatically activated upon exposure to ambient air and start its timing function. According to aspects of the disclosure, when the IV extension set 300, 310 is put in use, a peel off label or covering may be peeled off (e.g., removed) from the indicator portion 210, which then may be activated upon exposure to ambient air and start its timing function.

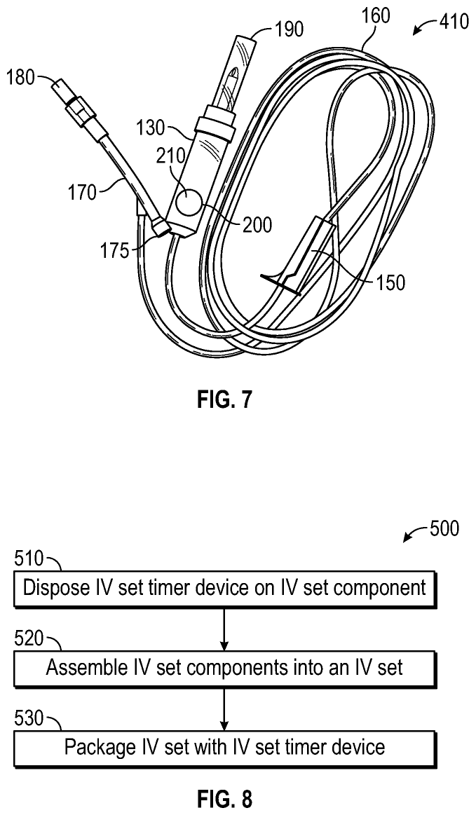

FIGS. 6 and 7 show example IV sets 400, 410 having different combinations of infusion components and IV tubing 160. For example, IV set 400 includes a drip chamber 130, a catheter 185, IV tubing 160, a flow controller 150 and a hanger 192, while IV set 410 includes a drip chamber 130 with a spike 190, a flow controller 150, a Y-site 170 with a needless port 175, a luer lock connector 180 and IV tubing 160. Both IV extension sets 400, 410 include an IV set timer device 200 disposed on the drip chamber 130 as a label, each IV set timer device 200 having an indicator portion 210 that changes color over time after being activated. Here, the IV set timer device 200 may be disposed on the drip chamber 130 because the drip chamber 130 is suitable (e.g., large enough, allowed to be partially obscured) to include the IV set timer device 200.

In aspects of the disclosure, an IV set timer device may be integral to an IV set component. For example, the IV set timer device 200 shown in FIGS. 6 and 7 may be formed as a section of the drip chamber 130 itself and/or the entire see through body of the drip chamber 130 may be include a timer device material that changes the color of the body over time while maintaining visibility into the drip chamber 130. Similarly, as section of IV tubing 160 or an entire length of IV tubing 160 may include a timer device material that changes the color of the IV tubing 160 over time while maintaining visibility into the IV tubing 160.

In aspects of the disclosure, any IV set component may include an integrally formed portion of timer device material, such as drip chamber 130, check valve 140, flow controller 150, tubing 160, Y-site 170, luer lock connector 180, catheter 185, spike 190, and the like. In any of the above, the IV set timer material portion of the IV set component may be activated by peeling off a cover or label, by being removed from airtight or vacuum sealed packaging, by being exposed internally and/or externally to a change in temperature, or any other suitable activation process.

In aspects of the disclosure, the IV set timer device 200 may be added to an IV set component during manufacturing and packaging of the IV set. In aspects of the disclosure, the IV set timer device 200 may be provided separately on a roll, a sheet, in individual packages or any other suitable storage device, where the clinician or user applies the IV set timer device 200 to some portion of the IV set when the IV set is put into use.

For example, a roll or sheet of IV set timer devices with cover labels may be on hand in the medical treatment area. Here, when a new IV set or even a new single IV set component is put into use, the clinician removes an IV set timer device 200 from the roll or sheet, thus exposing an adhesive back surface, and applies the adhesive surface to an IV set component, then peeling off a front cover label to activate the IV set timer device 200.

As another example, the IV set timer device 200 may be sealed in an airtight or vacuum sealed individual package. Here, when a new IV set or new single IV set component is put into use, the clinician removes an IV set timer device 200 from the sealed individual package, exposing the indicator portion 210 to the air to activate the IV set timer device 200, peels off a cover to expose an adhesive back surface, and applies the adhesive surface to an IV set component

In aspects of the disclosure, the IV set timer device 200 may be applied by an applicator device (e.g., applicator gun). For example, a handheld applicator device (e.g., like a tape gun or label applicator) may be used by the clinician to apply IV set timer devices 200 to IV set components as needed. Here, the applicator device may lift the IV set timer device 200 from a backing roll to expose an adhesive surface of the IV set timer device 200, and peel off a cover label from the indicator portion 210 to activate the IV set timer device 200 upon application from the applicator device to the IV set component. As another example, the applicator device may be a robotic or machine portion of a manufacturing system or assembly system, where the IV set timer device 200 is automatically applied to an IV set component during manufacturing, assembly or packaging of an IV set.

According to some aspects of the disclosure, a method 500 of making an IV set with an IV set timer device is shown in FIG. 8. In step 510, an IV set timer device (e.g., IV set timer device 200) is disposed on or attached to an IV set component (e.g., drip chamber 130, IV tubing 160). In aspects of the disclosure, the IV set timer device may be an adhesive label or tag that is attached to (e.g., stuck to) the IV set component. In aspects of the disclosure, the IV set timer device may be integrally formed with the IV set component.

An IV set is assembled in step 520. For example, various IV set components may be assembled into a complete IV set (e.g., IV set 400, 410). In aspects of the disclosure, the IV set is assembled first in step 520 and then the IV set timer device is disposed on the IV set component. In aspects of the disclosure, the IV set timer device is disposed on the IV set component and then the IV set is assembled.

In step 530, the IV set with IV set timer device is packaged. For example, the IV set with IV set timer device may be inserted into an airtight bag and the air in the bag is extracted. As another example, the IV set with IV set timer device may be vacuum sealed into a bag or other packaging form. In yet another example, the IV set with IV set timer device may be inserted into a non-airtight or vacuum sealed bag or package.

In one or more embodiments, an intravenous set timer device comprises an indicator portion configured to change color at one or more predetermined times upon activation, a legend portion, wherein the legend portion defines relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion.

In aspects of the disclosure, the indicator portion is a circle and the legend portion is disposed around a portion of an outer perimeter of the circle. In aspects of the disclosure, the legend portion comprises a plurality of sections, each section being a color that matches a color of the indicator portion at one of the predetermined times. In aspects of the disclosure, a printed word legend describes the time period is associated with each legend color. In aspects of the disclosure, an instruction portion is disposed around a portion of the outer perimeter of the circle, the instruction portion being a color that matches a color of the indicator portion at a predetermined expiration time. In aspects of the disclosure, a printed word instruction describes an action to be taken when the indicator portion changes to match the instruction portion color.

In aspects of the disclosure, the removable protective cover comprises a peel off label disposed on an outer surface of the indicator portion. In aspects of the disclosure, the removable protective cover comprises one of an airtight package and a vacuum sealed package containing the intravenous set timer device. In aspects of the disclosure, an adhesive coating is disposed on an engagement surface of the intravenous set timer device, the adhesive coating configured to attach the intravenous set timer device to an intravenous set component. In aspects of the disclosure, the intravenous set timer device is configured to be integrally formed with an intravenous set component.

In one or more embodiments, an intravenous set comprises a plurality of connected intravenous set components and an intravenous set timer device. The intravenous set timer device comprises an indicator portion configured to change color at one or more predetermined times upon activation, a legend portion, wherein the legend portion defines relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion.

In aspects of the disclosure, the indicator portion is a circle and wherein the legend portion is disposed around a portion of an outer perimeter of the circle. In aspects of the disclosure, the legend portion comprises a plurality of sections, each section being a color that matches a color of the indicator portion at one of the predetermined times. In aspects of the disclosure, a printed word legend describes the time period associated with each legend color. In aspects of the disclosure, an instruction portion is disposed around a portion of the outer perimeter of the circle, the instruction portion being a color that matches a color of the indicator portion at a predetermined expiration time, and a printed word instruction describes an action to be taken when the indicator portion changes to match the instruction portion color.

In aspects of the disclosure, the removable protective cover comprises one of a peel off label disposed on an outer surface of the indicator portion, an airtight package containing the intravenous set timer device, and a vacuum sealed package containing the intravenous set timer device. In aspects of the disclosure, the intravenous set includes intravenous tubing and the intravenous set timer device is attached to the intravenous tubing. In aspects of the disclosure, the intravenous set includes a drip chamber and the intravenous set timer device is disposed on the drip chamber. In aspects of the disclosure, the intravenous set timer device is integrally formed with one of the plurality of intravenous set components.

In one or more embodiments, a method of making an intravenous set with an intravenous set timer device comprises attaching the intravenous set timer device to an intravenous set component, wherein the intravenous set timer device comprises an indicator portion configured to change color at one or more predetermined times upon activation, a legend portion defining relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion, assembling a plurality of intravenous set components, including the intravenous set component with the attached intravenous set timer device, into an assembled intravenous set and packaging the assembled intravenous set.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous set timer device, comprising:
an indicator portion configured to change color at one or more predetermined times upon activation;
a legend portion, wherein the legend portion defines relationships between colors of the indicator portion and specific time periods; and
a removable protective cover configured to prevent exposure of the indicator portion to air when the removable protective cover is disposed on the indicator portion,
wherein the indicator portion is configured to be activated when the removable protective cover is removed from the indicator portion.

2. The intravenous set timer device of claim 1, wherein the indicator portion is a circle and the legend portion is disposed around a portion of an outer perimeter of the circle.

3. The intravenous set timer device of claim 2, wherein the legend portion comprises a plurality of sections, each section being a color that matches a color of the indicator portion at one of the predetermined times.

4. The intravenous set timer device of claim 3, further comprising:
a printed word legend describing the time period associated with each legend color.

5. The intravenous set timer device of claim 2, further comprising:
an instruction portion disposed around a portion of the outer perimeter of the circle, the instruction portion being a color that matches a color of the indicator portion at a predetermined expiration time.

6. The intravenous set timer device of claim 5, further comprising:
a printed word instruction describing an action to be taken when the indicator portion changes to match the instruction portion color.

7. The intravenous set timer device of claim 1, wherein the removable protective cover comprises a peel off label disposed on an outer surface of the indicator portion.

8. The intravenous set timer device of claim 1, wherein the removable protective cover comprises one of an airtight package and a vacuum sealed package containing the intravenous set timer device.

9. The intravenous set timer device of claim 1, further comprising:
an adhesive coating disposed on an engagement surface of the intravenous set timer device, the adhesive coating configured to attach the intravenous set timer device to an intravenous set component.

10. The intravenous set timer device of claim 1, wherein the intravenous set timer device is configured to be integrally formed with an intravenous set component.

11. An intravenous set, comprising:
a plurality of connected intravenous set components; and
an intravenous set timer device, comprising:
an indicator portion configured to change color at one or more predetermined times upon activation;
a legend portion, wherein the legend portion defines relationships between colors of the indicator portion and specific time periods; and
a removable protective cover configured to prevent exposure of the indicator portion to air when the removable protective cover is disposed on the indicator portion, wherein the indicator portion is configured to be activated when the removable protective cover is removed from the indicator portion.

12. The intravenous set of claim 11, wherein the indicator portion is a circle and wherein the legend portion is disposed around a portion of an outer perimeter of the circle.

13. The intravenous set timer device of claim 12, wherein the legend portion comprises a plurality of sections, each section being a color that matches a color of the indicator portion at one of the predetermined times.

14. The intravenous set timer device of claim 13, further comprising:
a printed word legend describing the time period associated with each legend color.

15. The intravenous set timer device of claim 12, further comprising:
an instruction portion disposed around a portion of the outer perimeter of the circle, the instruction portion being a color that matches a color of the indicator portion at a predetermined expiration time; and
a printed word instruction describing an action to be taken when the indicator portion changes to match the instruction portion color.

16. The intravenous set timer device of claim 11, wherein the removable protective cover comprises one of:
a peel off label disposed on an outer surface of the indicator portion;
an airtight package containing the intravenous set timer device; and
a vacuum sealed package containing the intravenous set timer device.

17. The intravenous set timer device of claim 11, wherein the intravenous set includes intravenous tubing and the intravenous set timer device is attached to the intravenous tubing.

18. The intravenous set timer device of claim 11, wherein the intravenous set includes a drip chamber and the intravenous set timer device is disposed on the drip chamber.

19. The intravenous set timer device of claim 11, wherein the intravenous set timer device is integrally formed with one of the plurality of intravenous set components.

20. A method of making an intravenous set with an intravenous set timer device, the method comprising:
attaching the intravenous set timer device to an intravenous set component, wherein the intravenous set timer device comprises an indicator portion configured to change color at one or more predetermined times upon activation, a legend portion defining relationships between colors of the indicator portion and specific time periods, and a removable protective cover configured to prevent exposure of the indicator portion to air when the removable protective cover is disposed on the indicator portion, wherein the indicator portion is configured to be activated when the removable protective cover is removed from the indicator portion;
assembling a plurality of intravenous set components, including the intravenous set component with the attached intravenous set timer device, into an assembled intravenous set; and
packaging the assembled intravenous set.
